(19) United States
(12) Patent Application Publication
(10) Pub. No.: US 2017/0239077 A1
(43) Pub. Date: Aug. 24, 2017

(54) NON-ROLLED ADHESIVE CONDOM AND ABBREVIATED CONDOM

(71) Applicant: John Ing Ching TANG, Sibu (MY)

(72) Inventor: John Ing Ching TANG, Sibu (MY)

(21) Appl. No.: 15/519,541

(22) PCT Filed: Oct. 16, 2014
(86) PCT No.: PCT/IB2014/065376

§ 371 Date: Apr. 15, 2017

(51) Int. Cl. A61F 6/04

(52) U.S. Cl. CPC A61F 6/04; A61F 2006/047; A61F 2006/049; A61F 2006/043; A61F 2006/048

(57) ABSTRACT

The present invention is a non-rolled adhesive condom with adhesive coating on its external surface supported by one or more releasing strips, a configuration that allows easy manufacture and deployment of said condom. Deployment involves donning the external surface of the closed end of said condom onto penis and pulling the releasing strips proximally and away from said condom, thereby inverting the adhesive coated external surface outside-in to adhere to penis. The condom may be constructed by sealing two or more membranes, even with the supporting films on. The tip of the closed end of condom has an adhesive-free area to serve as seminal receptacle. It can be opened to aid deployment of urinary condom catheter. Based on the above principle, a number of shortened non-rolled adhesive condoms (abbreviated condoms) comprising a seminal receptacle and adhesive flange(s) that wrap around the distal part of penis are also presented.

### TECHNICAL FIELD

The objective of the present invention is related to sexual barriers for prevention of fertilizing spermatozoa and sexually transmitted diseases (STD) during sexual intercourse. Particularly it relates to the protection of male organ to prevent exchange of body fluid by a thin flexible walled condom with adhesive. It is yet another objective of the present invention to provide an abbreviated condom that only covers the distal part of penis.

### BACKGROUND ART

Male latex condom is the commonest used barrier methods for the prevention of unintended pregnancies and sexually transmitted infections (STI). However it suffers from a number of drawbacks that deter its more widespread use. The main drawbacks are reduced tactile/thermal sensation and easy slippage/ leakage. The sensation issue is due to thickness of the condom and strangulating grip of condom on penis, while the slippage/leakage problem is encountered whenever penis is not tumescent enough to keep the condom stretched, which happens not only after ejaculation, but often along the course of sexual act, both in situ and ex situ.

Multiple prior art have attempted to solve the above issues by developing thinner condom as well as using adhesive to anchor condom onto penis.

Ultra-thin condoms (of less than 0.05 mm) can be produced by using biological (such as beef tendon) or synthetic (such as plastic polymers) flexible membranes. Through the use of modern technology, thinner membrane can now be developed without significantly compromising the strength of the material.

By using adhesive on condom, the issues of strangulating grip on penis and condom slippage/leakage can be overcome regardless of the rigidity of penis. Furthermore, since adhesive eliminates friction between condom and penis, the condom is less likely to break, thereby allowing thinner material to be used. This in turn results in providing better tactile and thermal sensation for the user.

However, addition of adhesive to condom presents a challenge in both its manufacture and deployment.

U.S. Pat. No. 4,638,790 discloses a condom in which the hood, initially in a rolled-up configuration, has an outer member which transfers adhesive onto the inner surface of the condom as the hood is unrolled onto penis. The outer member is removed after the condom is in place. The main problem with such a configuration is that the user's hands are in direct contact with the adhesive surface during unrolling of the condom, thereby affecting the integrity of pressure sensitive adhesive.

In a patent granted to Attila (U.S. Pat. No. 7,963,285) it is provided a rolled condom with adhesive on the inner surface supported by a cylindrical releasing strip. The cylindrical releasing strip extends beyond the open end of condom and flares out circumferentially to form a graspable deployment rim/ring. The condom together with releasing strip within it is rolled up during assembly. It is unrolled onto penis by pulling the releasing strip proximally using the rim/ring. While donning a rolled non adhesive condom by direct unrolling action is relatively easy, donning a rolled adhesive condom by pulling on the releasing strip is more difficult as a lot more force is required at unrolling an adhesive entity let alone pulling it. Moreover the manufacturing of rolled adhesive condom that involves application of relatively non elastic releasing strip on an adhesive coated elastic condom followed by rolling is technically difficult.

There is therefore a need for an ultra-thin non-rolled adhesive condom that is cheap to produce and easy to deploy by users for prevention of unintended pregnancy and sexually transmitted infections.

It is yet another objective of the current invention to provide abbreviated condoms that are based on the same principle.

Abbreviated condom is a condom that attaches solely to distal end of penis, thereby leaving a greater portion of penile shaft exposed for more direct contact between sexual partners. It is well known that the distinctive frusto-conical dilating probe-like shape of corona glandis and deep coronal sulcus of penis are the main body parts contributing to the pleasure of sexual experience. Unfortunately donned conventional condom negates such nature's intended design by constricting the corona glandis as well as obliterating the coronal sulcus. Abbreviated condom that covers only the distal part of glans penis can therefore overcome such problems. U.S. Pat. No. 4,869,269 and U.S. Pat. No. 6,035,854 show contraceptive-prophylactic devices made out of a thick material mold that use adhesives to stick on the tip of glans penis. U.S. Pat. No. 5,421,350 shows an unrolled condom that through adhesive means covers only the head of the penis. U.S. Pat. No. 6,145,507, U.S. Pat. No. 4,821,742, and U.S. Pat. No. 3,677,225 show abbreviated condoms that use adhesive that sticks to the tip of glans penis. U.S. Pat. No. 6,148,819 shows a method and kit containing a liquid adhesive for attaching a condom to the penis and a liquid solvent for removing the condom from the penis. All of these prior art describe the same concept of an abbreviated condom comprising an adhesive-free seminal receptacle with encircling adhesive flange that sticks onto the distal part of penis. However, none of these inventions are as simple to manufacture or user friendly as the present invention.

### DETAILED DESCRIPTION OF THE INVENTION

In accordance with the present invention there is provided a thin flexible adhesive condom that is to be worn by a male person to prevent the transmission of infections and sperm during sexual intercourse. The condom is designed to adhere to penis regardless of its degree of rigidity. It is specifically constructed in a non-rolled configuration for easy manufacture and deployment. It is another objective of the present invention to provide an abbreviated or micro condom that solely covers the distal part of penis to minimize the barrier between the sexual partners.

The non-rolled condom (FIG. 1) has at least a part of the external surface coated with hypoallergenic waterproof adhesive that is supported by one or more backing membranes (or releasing strips). The releasing strip may extend beyond the adhesive coating to form a loose end that can be used as pulling tag for condom deployment. The method of construction of condom may include, but not limited to, dipping, molding and sealing of two or more ultra thin membranes. Different flexible, thin and impermeable membrane can be used for making the condom, for instance rubber latex or other biologically acceptable plastic films, such as polyurethane, polyethylene, polypropylene, polyester, nylon, polyvinyl chloride, bioplastics, nitrile and so on.

In a preferred embodiment, the non-rolled adhesive condom is constructed by using a loose fitting condom which is applied on the external surface with adhesive and two flexible releasing strips that has loose ends extending beyond the adhesive coating (FIG. 1). To deploy the condom, the user opens up the open end of the condom and caps the loose fitting condom onto penis by pointing the external surface of the closed end of condom towards glans penis. This is followed by expelling the air from the closed end of condom and pulling off the loose ends of releasing strips proximally towards his body. The pulling action will invert the condom outside-in, internalizing the adhesive coated external surface of the condom, thereby adhering condom to penis (FIGS. 2 to 4). The wrinkles thus produced by the loose fitting condom will not present as a problem as the condom is made of thin flexible material. In fact the wrinkles at the open end of the condom can be used as tags to remove the condom after use. The said loose fitting condom thus opens up the possibility of manufacturing a single sized condom that fits all.

In another embodiment, there is only one releasing strip. The releasing strip may be cylindrical, non cylindrical, or polygonal. It may be provided with breaking means, such as one or more tearing lines, during manufacturing and they are broken to aid removal of releasing strip during deployment.

A preferred process of manufacturing the non-rolled adhesive condom consists of a condom produced by dip molding, coating of adhesive on its external surface followed by application of releasing strip(s). Alternatively the adhesive coating may be transferred to the external surface of condom by using releasing strip(s) that is/are pre coated with adhesive.

Another preferred process of manufacturing the non-rolled adhesive condom consists of sealing of two thin flexible membranes in the designated shape of the said condom, application of adhesive coat and releasing strip on the external surface of one membrane with simultaneous, or followed by, application of adhesive coat and releasing strip on the external surface of the other membrane. The final product is produced by cutting the above assembly into the designated shape.

A variety of sealing modalities may be used to seal two thin membranes in constructing the said condom. These may include, but not limited to, heat, chemical, ultrasound, radio frequency, electrical, light and mechanical means. When energy source is used, sealing of membranes may be performed by applying said energy directly on the membranes or through the supporting films. As thin membrane is often presented with a less flexible supporting film of a different material attached to its surface for easy handling, said membranes can be sealed by applying sealing energy through the supporting film(s). An example is polyurethane membrane which is underpinned by polyethylene terephthalate supporting film. Since polyurethane membranes have lower melt strength than polyethylene terephthalate supporting films, the former may be heat sealed by applying heat source through the later without sealing effect on the later. The heat strength should therefore be just enough to seal the two inner membranes without sealing the inner membranes to the outer supporting films.

In another embodiment, the condom has added textures, colours, drawings, wordings, ridges nubs and/or studs to enhance sexual gratification. The non adhesive surface may be coated with lubricant that becomes slippery at the addition of water or other liquid.

In another embodiment, the releasing strip(s) and/or supporting film(s) have added textures, colours, drawings and/or wordings to enhance sexual gratification.

In another embodiment, the open end of the condom has an irregular edge to suit individual preference.

In another embodiment, there is an uneven distribution of adhesive on the condom. In particular the adhesive coating is stronger towards the open end of condom to prevent condom slippage, and weaker towards the closed end to avoid excessive exposure of adhesive to the sensitive glans penis. The strength of adhesive coating can be qualitative and/or quantitative. For example, the adhesive coating away from the open end of condom may be of a weaker type, thinner or only partially coated.

In another embodiment, the open end of condom has one or more adhesive-free loose tags (isolated flare out portions) acting as pulling tags in aiding removal of condom. In another embodiment the open end of condom is adhesive free so that the loose open end can used as pulling tag to peel it off from penis after use.

The non-rolled adhesive condom may be used as a means to secure urinary condom catheter by simply inserting the catheter's connecting tubing through the cut tip of the closed end of condom, donning the assembly onto glans penis and deploying the condom as mentioned above (FIG. 5). By attaching the condom catheter to penis, the thin adhesive condom thus provides a leak proof means of deploying the urinary condom catheter on a flaccid penis (FIG. 5a), an often difficult task to achieve for the existing relatively thicker adhesive urinary condom catheters. The opening at the closed end of condom can be made by cutting or by being left open during manufacturing.

Another object of the present invention is to provide an abbreviated condom or micro condom that covers solely the distal portion of penis by shortening the shaft portion of said condom. Such configuration has the advantage of minimizing the barrier between sexual partners while achieving contraceptive effect. In fact the shaft portion of condom can be shortened to the extent of just comprising a seminal receptacle and its surrounding flange that adheres to distal part of glans penis (FIG. 6).

A number of preferred embodiments may be provided for the said abbreviated condom. One embodiment is prepared as described above with adhesive coating on the external surface and it is inverted outside in during deployment (as shown in FIG. 1-4).

In another embodiment, the opposing walls of the shortened non-rolled adhesive condom are opened longitudinally, leaving the closed end of the condom and seminal receptacle intact (FIG. 7). The two adhesive flanges thus created, and supported by releasing strips, are deployed onto glans penis with the seminal receptacle inverted (FIG. 8). The use of flanges helps to ensure that the open end of seminal receptacle is positioned precisely over the external urethral meatus (say, by applying the flanges one at a time) and to facilitate a wrinkle free deployment of said condom on the frusto-conical glans penis.

In another embodiment, the abbreviated condom may be manufactured by sealing two thin membranes in an omega or similar shape, wherein the omega dome becomes seminal receptacle while the two flanges that extend from omega base (FIG. 10) become the body of abbreviated condom. Adhesive is coated on the internal surfaces of both flanges, supported by folded releasing strips (backing membranes) (FIG. 10). At deployment, the releasing strips are removed to allow adhesive flanges to wrap around glans penis (FIG. 10a). Alternatively adhesive is coated on the external surface of said flanges, supported by releasing strips, and said seminal receptacle is inverted before deployment.

During production, the order of process of manufacture may vary according to the method of sealing. For example, while the above mentioned process using two membranes is applicable for heat sealing, sealing by using adhesive (FIG. 9) may preferably involve coating of adhesive onto internal surfaces of the two individual membranes (each with a suitably shaped adhesive-free area as seminal receptacle), application of releasing strips to the adhesive flanges, apposition of said two membranes (with said adhesive-free areas facing and coincide with each other) and cutting it into designated shape of abbreviated condom. At deploying the abbreviated condom, the two adhesive flanges will adhere to one another as well as to glans penis (FIG. 9a). The said adhesive free area for seminal receptacle may also be created by applying a suitably shaped non adhesive ultra thin membrane on the adhesive coated membrane base. The resulting seminal receptacle will thus have a wall comprising double layers of membranes, thereby rendering it less likely to break. The abbreviated condom thereof may be provided with one or more external supporting films made of less elastic material to aid in its manufacture, to serve as protective cover(s), and to facilitate deployment.

In another embodiment, a single elongated membrane that folds on itself is used to create a configuration of double membranes (FIG. 11). In this case the process of preparing the abbreviated condom may consist of adhesive coating at the outer edges of a thin elongated membrane, which is disposed with one or more supporting films on its back, with a releasing strip strategically position at each adhesive end covering up a portion of the substantially centrally located adhesive free area. At folding the membrane, the adhesive edges at the segment near the fold will adhere to each other to form a seminal receptacle (FIG. 11). The condom is deployed onto glans penis by pulling off both releasing strips, smoothing both adhesive flanges onto glans penis and removing the external supporting film(s) (FIG. 11b). During production, the said supporting film may be pre treated with a folding line or grove, or a perforation line for easy and precise folding of said elongated membrane. It is important to note that as it is preferable that the open end of seminal receptacle thereof has an adhesive-free surrounding area in a shape and size sufficient to cover external urethral meatus, it is necessary for each releasing strip to cover a portion of the centrally located adhesive free area during assembly.

The flange(s) and seminal receptacle may be of varying size and shape to cater for variations in individual's penile size, seminal volume and preference. Notably the posterior flange may need to be larger to compensate for the irregular surface on the posterior aspect of glans penis.

### DESCRIPTION OF THE DRAWINGS

The inventions are herein described, by way of example only, with reference to the accompanying drawings, wherein:

FIG. 1: Sectional view of preferred embodiment of non-rolled condom 1 with adhesive coating 2 on external surface of condom supported by two releasing strips 3. The releasing strips 3 may have loose ends 3a to be used as pulling tags to facilitate deploying said condom 1. In the preferred embodiment the adhesive-free closed end 4 of condom 1 has a seminal receptacle 4a. The arrow denotes the direction of donning the condom onto glans penis.

FIG. 1a: Perspective of the said condom as in FIG. 1 before deployment.

FIG. 1b: Perspective view of the condom as in FIG. 1 opened up to be ready for deployment.

FIG. 2: Sectional view of said condom 1 donning onto penis 5. As the external surface of closed end 4 of condom 1 dons onto penis 5, the condom 1 is inverted outside-in. The arrows denote the direction of condom deployment. The process internalizes the adhesive coated 2 external surface of condom 1 and adheres the condom 1 to penis 5.

FIG. 3: As per FIG. 2 wherein the said condom 1 is further deployed onto penis 5.

FIG. 4: Sectional view of the adhesive condom 1 fully deployed onto penis 5. The adhesive coated external surface of condom 1 is fully internalised to adhere to penis 5.

FIG. 5: Sectional view of urinary condom catheter 13 with its connecting tube 13a inserted through an opening 1c at the closed end of condom 1. The opening 1c can be created by cutting the closed end 4 of condom 1, or by being left opened at manufacture. The condom 1 has its external adhesive 2 surface inverted outside in to adhere to condom catheter 13.

FIG. 5a: Sectional view of urinary condom 13 duly deployed onto penis 5 with the aid of adhesive condom 1, which adhered to both the condom 1 and penis 5 to give a leak proof seal.

FIG. 6: Sectional view of a preferred embodiment of adhesive abbreviated condom deployed onto penis 5. The abbreviated condom only comprises a seminal receptacle 4a and an adhesive flange 1b that covers solely the distal part of penis 5a. The adhesive abbreviated condom may simply be a shortened version of said non-rolled adhesive condom.

FIG. 7: Perspective view of a shortened version of said no rolled adhesive condom 1 with opposing side wall opened, while leaving the closed end 4 and seminal receptacle 4a intact, thereby creating two adhesive flanges 1b supported by releasing strips 3.

FIG. 8: Perspective view of the abbreviated condom as in FIG. 7, wherein the external adhesive coating 2 is partially exposed as the condom is opened up for deployment. In this configuration, the two flanges 1b are jointed at the closed end 4 and at the dome of seminal receptacle 4a. Loose ends 3a of the releasing strips 3 are used as pulling tags to deploy the condom.

FIG. 8a: Perspective view of the abbreviated condom as in FIG. 8 duly deployed onto glans penis 5a. The seminal receptacle 4a is inverted during deployment. Note that the area immediately surrounding the open end of seminal receptacle 4a, demarcated by adhesive margin 2a, is adhesive-free to accommodate external urethral meatus 5b.

FIG. 9: Perspective view of a preferred embodiment of abbreviated condom comprising two ultra thin membranes 1a adhere 2 together, with a substantially centrally located adhesive-free area demarcated by adhesive margin 2a, to form seminal receptacle 4a at the upper portion, and two adhesive flanges 1b supported by releasing strips 3 at the lower portion. The two releasing strips 3 are folded near the junction 2b between the upper and the lower portions of abbreviated condom to create two respective loose ends 3a that serve as pulling tags.

FIG. 9a: Perspective view of the abbreviated condom as in FIG. 9 duly deployed onto glans penis 5a. Deployment of the abbreviated condom thereof consists of expelling the air in the seminal receptacle 4a, placing the open end 4c of seminal receptacle 4a over the external urethral meatus 5b, and removing releasing strips 3 by pulling off their respective loose ends 3a. The exposed adhesive coating on the flanges 1b will, on smoothing against the distal end of penis, adhere to each other as well as glans penis 5a to form a leak proof seal. Note that said junctions 2b have now elongated and shifted into the said lower portion as the membranes 1a at said upper portion merge with flanges 1b to conform to the shape of glans penis 5a.

FIG. 10: Perspective view of another preferred embodiment of abbreviated condom. It is constructed by sealing two ultra thin flexile membranes in an omega or similar shape wherein the omega dome 6 contains the seminal receptacle 4a, and the omega base 6a together with two extended flanges 1b become the body of said abbreviated condom, thus resembling a configuration of a shortened non-rolled adhesive condom with opposing sides of the lateral wall being cut open as above mentioned. The internal surfaces of the flanges 1b are coated with adhesive 2 with the support of two releasing strips 3, both of which are folded near the omega base 6a with loose ends 3a serving as pulling tags for easy deployment of said abbreviated condom. The arrows denote the direction of pulling of loose ends 3a of releasing strips 3 during deployment. The open end 4c of the seminal receptacle 4a and its immediate surrounding area demarcated by adhesive margin 2a, which are adhesive free, are to be placed at external urethral meatus during deployment.

FIG. 10a: Perspective view of the abbreviated condom as in FIG. 10 duly deployed onto glans penis 5a. The air in the seminal receptacle 4a is expelled and with the removal of releasing strips 3 by pulling off the loose ends 3a, both flanges 1b are adhered onto glans penis 5a. The omega base 6a that connects the two flanges 1b thereof becomes part of the adhesive abbreviated condom. The adhesive-free open end 4c of the seminal receptacle 4a and its immediate adhesive-free surrounding area join up with external urethral meatus 5b to form a continuous passage for seminal fluid to be collected in the seminal receptacle 4a during ejaculation.

FIG. 11: Perspective view of an abbreviate condom prepared by folding. It comprises an elongated thin membrane, underpinned by a supporting film, which is coated with adhesive solely on its outer edges, with a releasing strip (backing membrane) disposed at each adhesive end of said membrane. Prior to deployment, the assembly is folded 8 with both releasing strips 3 partially pulled off towards each respective end to line 2b, where a portion of adhesive free area is still covered by releasing strip 3, to expose a segment of adhesive edges 1a near the fold 8 so that the edges 1a adhere to each other at folding, and thereby creating a seminal receptacle 4a. The folded configuration now resembles the abbreviated condom as shown in FIG. 9 ready for deployment. Note that the external supporting film(s) which are to be removed after deployment of said abbreviated condom are not shown in the figure.

FIG. 11a: Perspective view of abbreviated condom as in FIG. 11 dully deployed onto distal end of penis as described in FIG. 9a.

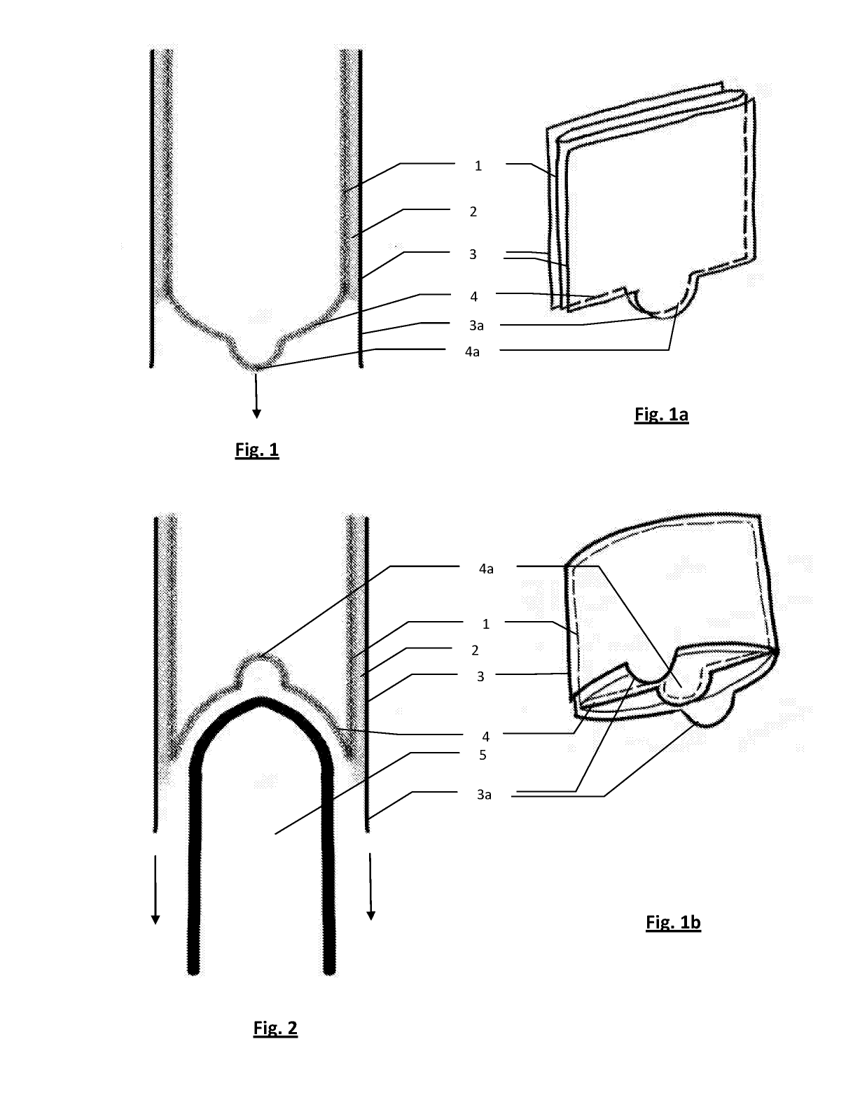

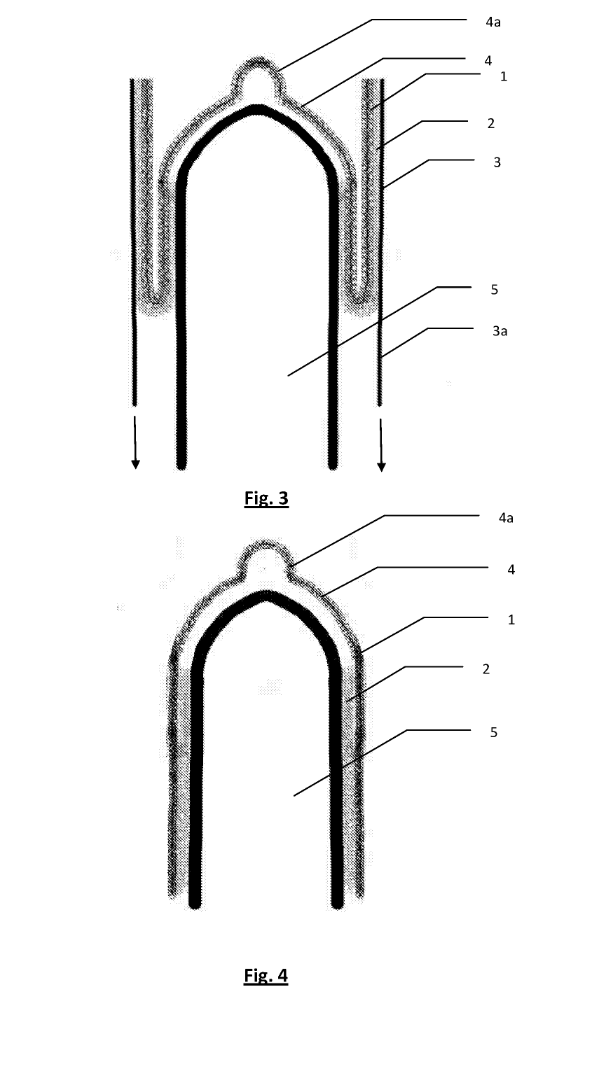

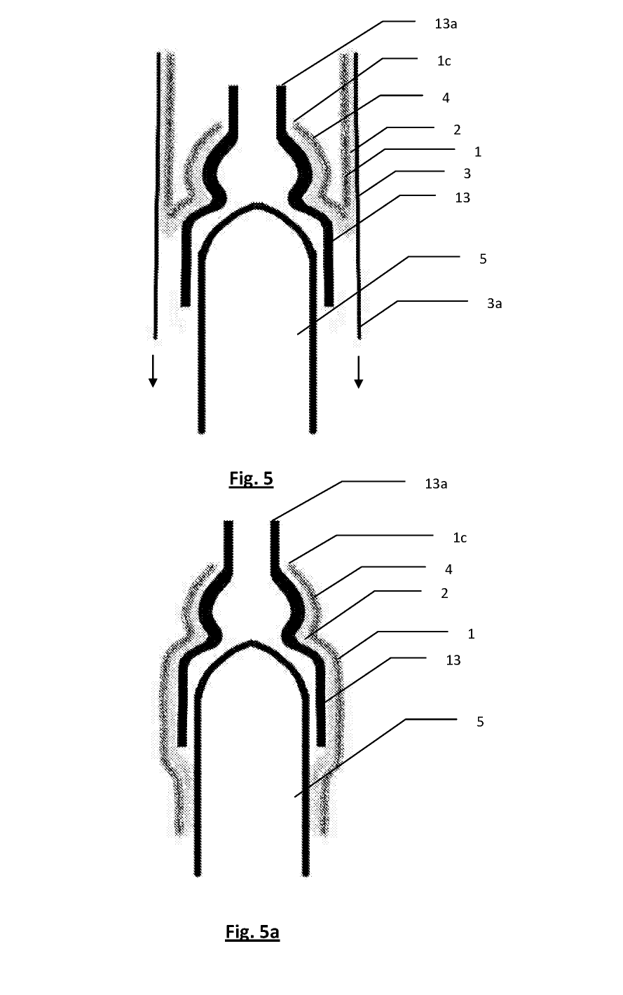

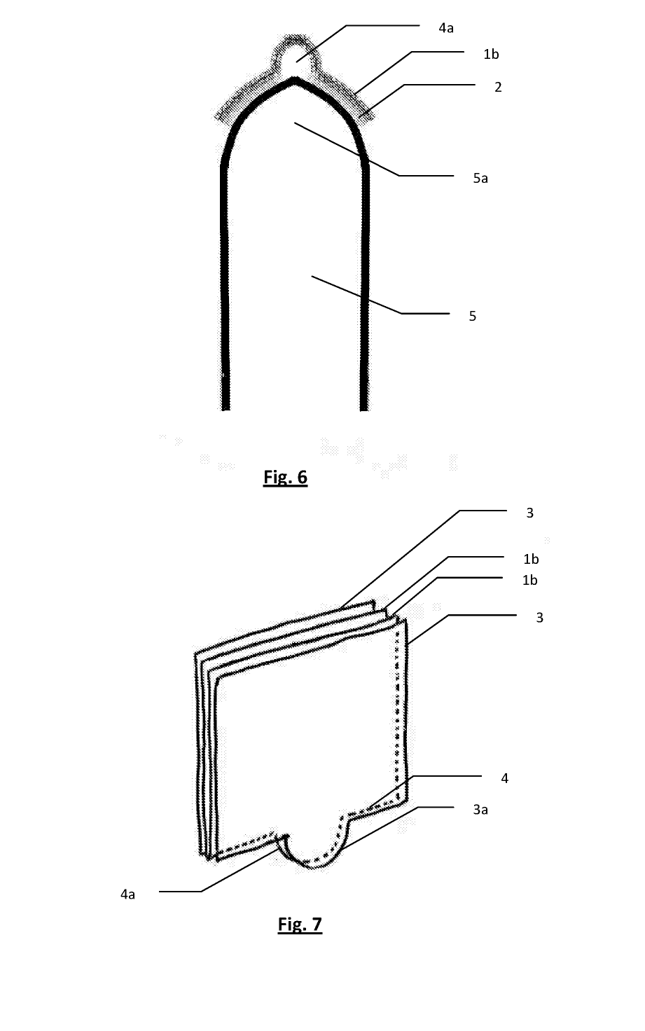

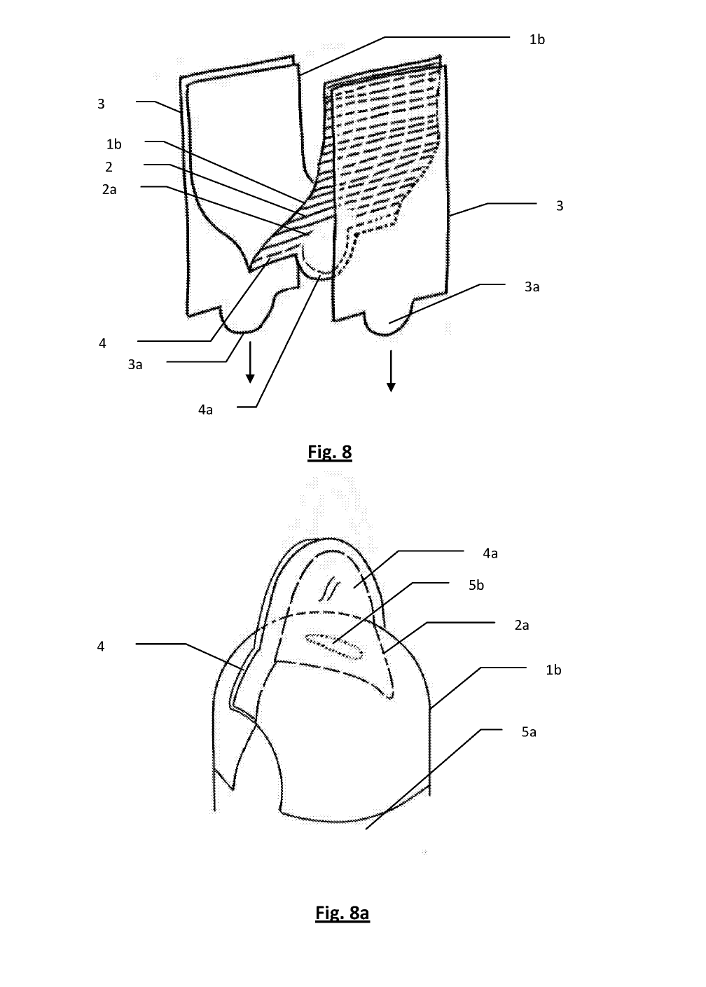

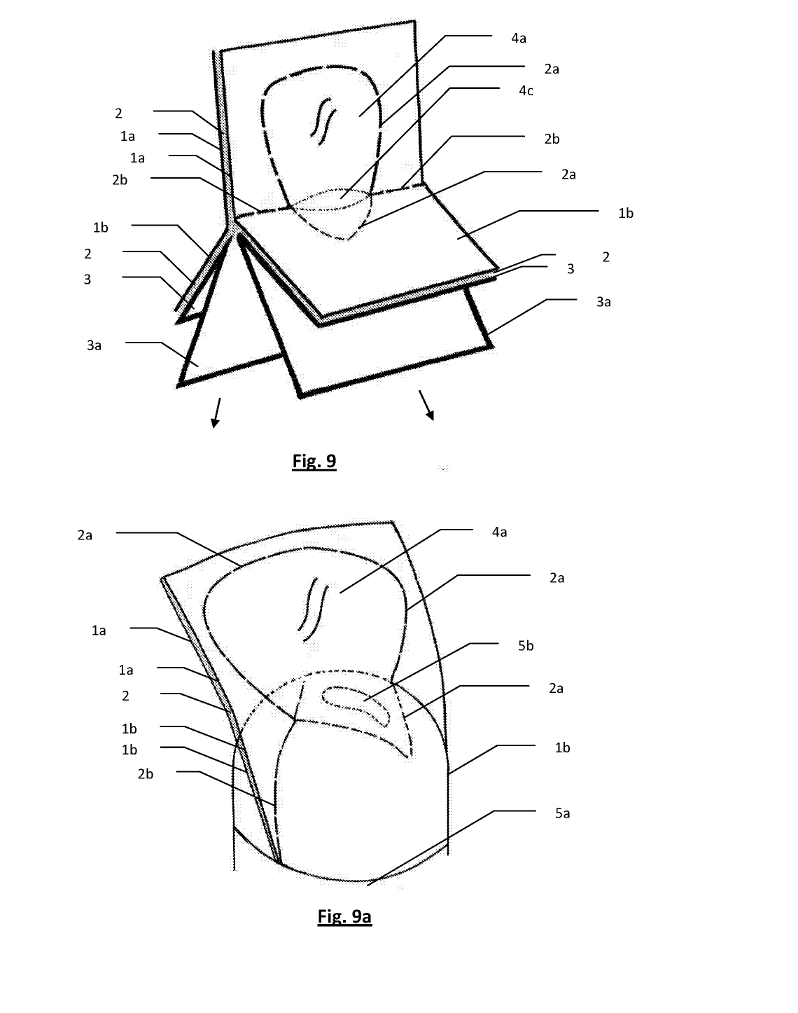

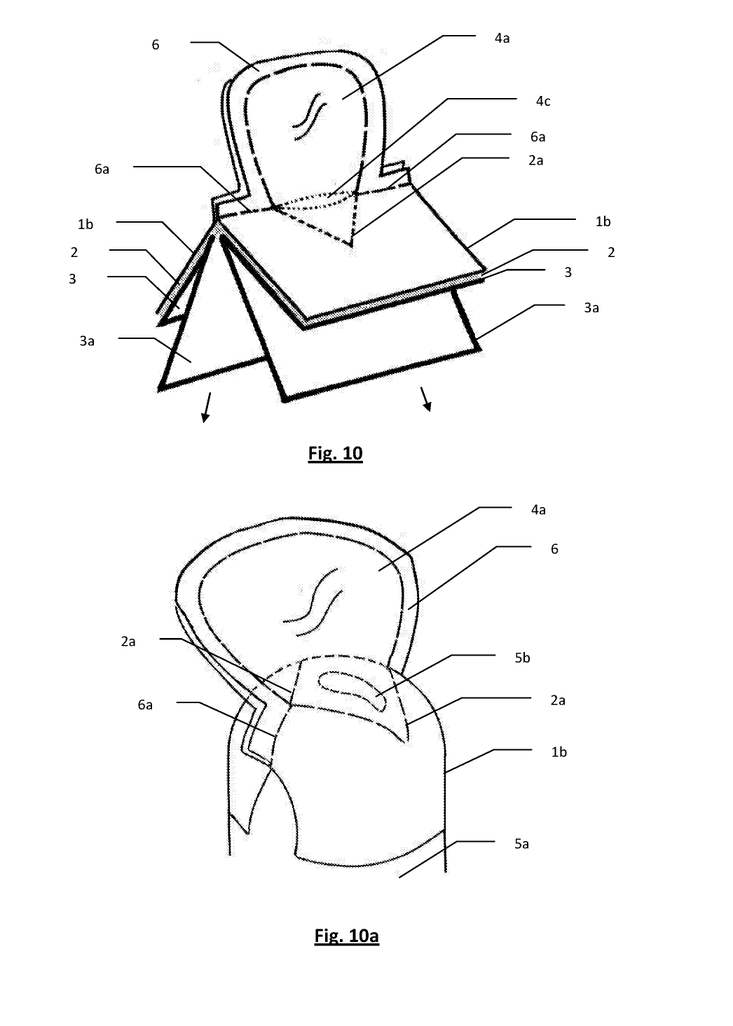

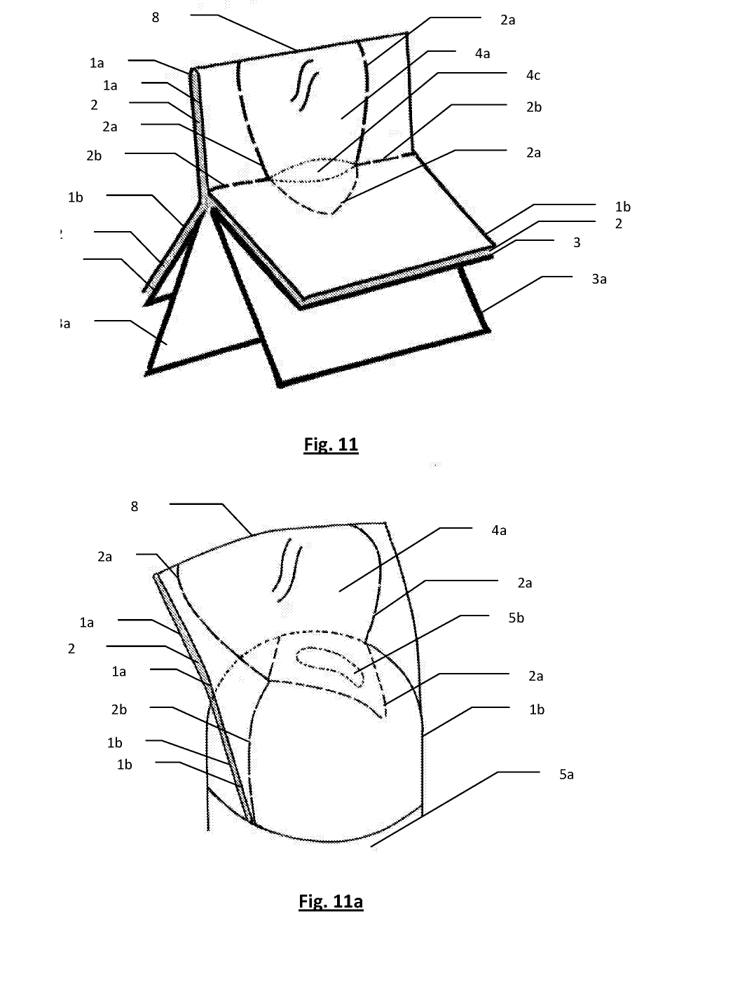

1. A non-rolled condom with a closed end and an open end, wherein
a) the external surface of said condom is at least partially coated with adhesive;
b) one or more releasing strips are disposed on said adhesive coated external surface;
c) deployment of said condom involves donning the external surface of the closed end of condom onto glans penis followed by advancing the condom with attached releasing strip(s) proximally towards penile shaft until the releasing strip(s) come off the condom completely. The condom is thereby inverted outside-in to adhere onto penis.

2. The condom as in claim 1, wherein the closed end of said condom has adhesive free pointed area(s) or protrusion(s) as seminal receptacle(s).

3. The condom as in claim 1, wherein the releasing strip(s) have loose end(s) acting as pulling tag(s) to deploy the condom.

4. The condom as in claim 1, wherein the condom is made by sealing two or more thin flexible impervious membranes.

5. A method of sealing two thin flexible impervious membranes, each disposed with a less flexible outer supporting film, wherein energy source is applied through said outer supporting film(s).

6. The condom as in claim 1, wherein the processes of manufacturing are:
a) Sealing of two membranes in the preferred shape of said condom;
b) Application of adhesive and releasing strip on the external surface of one membrane;
c) Simultaneous or followed by application of adhesive and releasing strip on the external surface of the other membrane;
d) Cutting the above assembly into final product.

7. The condom as in claim 1, wherein the closed end of said condom is cut open, or left open at manufacture, to be used for securing urinary condom catheter onto penis.

8. The condom as in claim 1, wherein the condom is shortened to comprise only a seminal receptacle with an adhesive membranous flange to cover solely the distal part of penis.

9. The abbreviated condom as in claim 8, wherein the opposing wall of condom shaft is divided or left opened to create two flanges that adhere to distal part of penis.

10. An abbreviated condom comprising two thin impervious membranes adhered together with substantially centrally located adhesive free area (as seminal receptacle), and two adhesive flanges (as body of abbreviated condom) that are supported by releasing strips.

11. An abbreviated condom comprising two thin impervious membranes sealed in a omega or similar shape, wherein the omega dome becomes the seminal receptacle, while the omega base with two extended flanges, coated with adhesive on the internal surfaces and supported by releasing strips, becomes the body of said abbreviated condom that adheres to the distal part of penis.

12. The abbreviated condom as in claim 11, wherein the two flanges extending from the omega base are coated with adhesive on the external surface, supported by releasing strip(s).

13. The abbreviated condom as in claims 10 and 11, wherein the external surface of the abbreviated condom is disposed with one or more supporting films for ease of manufacture and deployment.

14. A thin elongated impervious membrane, underpinned by one or more supporting films, which is coated with adhesive on the outer edges, thereby creating a substantially centrally located adhesive free area(s), with each end disposed with a releasing strip (backing membrane) that overlaps a portion of said adhesive free area(s).

15. The assembly as in claims 14, wherein it is folded to turn into an abbreviated condom.

16. The abbreviated condom as in claims 8, 9, 10, 11, 12, 13 and 15, wherein both the flange(s) and seminal receptacle may be of varying sizes and shapes to cater for variation in individual's penile size, seminal volume and preference.

17. The condom as in claims 1, 8, 9, 10, 11, 12, 13 and 15, wherein the condom has added lubricant, flavours, perfumes, textures, colours, drawings, wordings, ridges, nubs and /or studs.

18. The condom as in claims 1, 8, 9, 10, 11, 12, 13 and 15, wherein the releasing strip(s) and/or supporting film(s) have added perforations, folding grooves, textures, colours, drawings, markings and /or wordings.

19. The condom in claims 1, 8, 9, 10, 11, 12, 13 and 15, wherein there are uneven and/or different adhesive coatings on different parts of the condom surface.

20. The condom as in claims 1, 8, 9, 10, 11, 12, 13 and 15, wherein the edges of condom have adhesive free area(s) acting as handle(s) to aid the removal of condom.
